(19) United States
(12) Patent Application Publication
(10) Pub. No.: US 2005/0002993 A1
(43) Pub. Date: Jan. 6, 2005

(54) Confectionery products for delivery of pharmaceutically active agents to the throat

(76) Inventors: Paul Laurence Goggin, Bradford on Avon (GB); Caren Louise Cadra, Basking Ridge, NJ (US)

Correspondence Address: PFIZER, INC., 201 TABOR ROAD, MORRIS PLAINS, NJ, 07950, US

(21) Appl. No.: 10/838,044

(22) Filed: May 3, 2004

Related U.S. Application Data
(60) Provisional application No. 60/467,454, filed on May 2, 2003.

(51) Int. Cl. A61K 9/68

(52) U.S. Cl. 424/440

(57) ABSTRACT

A confectionery product for delivering at least one pharmaceutically active agent which includes a hard outer shell and a core comprised of a core material which is or forms a liquid-like to a gel-like substance in the oral cavity and is capable of delivering pharmaceutically active agents to infected and/or irritated tissues of the throat.

### PRIORITY INFORMATION

This application claims priority to U.S. provisional application No. 60/467,454, filed May 2, 2003.

### FIELD OF THE INVENTION

The present invention is generally directed to confections also referred to herein as confectionery products for delivery of active agents to the throat. The confections include a core and a shell surrounding the core so that the core is generally centrally positioned therein. The composition of the core and shell make the confection uniquely suited for the targeted delivery of active agents to the throat.

### BACKGROUND OF RELATED TECHNOLOGIES

There are many causes of throat problems from colds, irritation and the like. Such throat problems may be due to an infection of the lining of the mouth and throat caused by bacteria or viruses or to irritated tissues caused by irritants such as cigarette smoke, alcohol, pollutants, air conditioning and the like.

Products employed to provide instantaneous symptomatic relief from such throat conditions includes throat drops, lozenges, gargles and throat sprays. Lozenges are very popular for the temporary symptomatic relief of throat problems caused by infection and/or irritation. Gargles are less popular than lozenges because many people find gargling difficult and/or inconvenient because gargling cannot be performed in all venues. While throat sprays may be effective throat soothing vehicles, they are less popular since some people find throat sprays difficult to take because they may initiate an involuntary gagging or choking response.

Lozenges and related types of products such as sucking candies, lollypops and the like may be useful vehicles for delivering an active agent to the buccal cavity. However, lozenges fall short of delivering the active agents to the desired location of the throat due to the manner in which they are constructed.

One of the problems associated with standard lozenges is exemplified by the use of local anesthetic active agents. Quite often, a lozenge containing a local anesthetic, delivers the local anesthetic to the front portion of the oral cavity causing the tongue and/or the top of the mouth to feel numb. Thus, much of the local anesthetic is actually delivered to a portion of the oral cavity that is not in need of relief instead to the desired tissues of the throat which are infected and/or irritated.

Further, lozenges containing other active agents, such as antibacterial agents, are not effectively delivered to the throat in sufficient concentration to realize the maximum potential of the active agent and thereby obtain the desired relief in as short a period of time as possible. Furthermore, currently available lozenges are not be capable of containing multiple ingredients that are incompatible with each other which would lead to an unstable lozenge. Still further, currently available lozenges are not be able to deliver multiple ingredients at different times to the throat in one dosage form.

Accordingly, it would be desirable to provide a lozenge that is capable of delivering active agents to targeted areas of the throat. Further, it would be desirable to provide a lozenge that is capable of delivering two incompatible ingredients in one stable dosage form. Still further it would be desirable for a lozenge to deliver multiple ingredients at different stages.

### SUMMARY

One embodiment of the present invention provides a confectionery product for the delivery of at least one pharmaceutically active agent to a targeted tissue of the throat comprising a core comprising a carrier material; the carrier material when exposed to the oral cavity being in liquid form suitable for contacting the targeted tissues of the throat, and at least one first pharmaceutically active agent suitable for treating said targeted tissues; a shell comprising a solid material suitable for dissolving in the oral cavity the core positioned within the shell; and the core being substantially void of gas.

In another embodiment there is provided a method of producing a confectionery product for delivering at least one pharmaceutically active agent to targeted tissues of the throat, the confectionery product comprising a core and a shell, the core containing a carrier material having a physical form ranging from a liquid to a solid, the carrier when exposed to the oral cavity being in a liquid form for contacting the targeted tissues of the throat, and at least one first pharmaceutically active agent suitable for treating the targeted tissues, the shell comprising a solid material suitable for dissolving in the oral cavity and optionally comprising at least one second active agent, the method comprising combining the carrier material with the first active agent to form a core material in a first vessel; removing any gas contained within the core material; forming a shell material containing the optional second active agent in a second vessel; injecting an interruptible stream of the core material into a conduit while simultaneously injecting a continuous stream of the shell material external of the core material; and intermittently ejecting the combined stream in the form of the confectionery product. The combined streams are ejected in the form of the confectionery product into a tray containing a plurality of individual confection receiving compartments and allowing the confectionery product to cool therein to ambient temperatures. The core is visible through the shell.

In yet another embodiment, there is provided a confectionery product for the delivery of at least two pharmaceutically active agents to a targeted tissue of the throat comprising a core comprising a carrier material; the carrier material when exposed to the oral cavity being in liquid form suitable for contacting the targeted tissues of the throat, and at least one first pharmaceutically active agent suitable for treating the targeted tissues; a shell comprising a solid material suitable for dissolving in the oral cavity; the shell comprising at least one second pharmaceutically active agent; the core positioned within the shell; and the core being substantially void of gas.

### DETAILED DESCRIPTION

In one embodiment of the present invention, there is provided a confectionery product, such as a lozenge, that is capable of delivering active agents to targeted areas of the throat. In another embodiment of the present invention, there is provided a lozenge that is capable of delivering two incompatible ingredients in one stable dosage form.

In another embodiment, there is provided a lozenge that is capable of delivering multiple ingredients in different phases. More particularly, the lozenge may contain active ingredients in the shell and the core that are different so that the shell first delivers an active ingredient in the shell and once the shell has been dissolved, the center delivers an active ingredient which may act synergistically or complementary. For instance, a lozenge may have a shell containing nicotine and a center containing antimicrobial and/or breath freshening ingredients such as the essential oils as found in Listerine Pocket Paks manufactured by Pfizer Inc. This lozenge advantageously delivers a dose of nicotine first and followed by a antimicrobial amount of the essential oils to leave a refreshing taste in the mouth absent nicotine. In another instance, the active in the shell may prime the mouth to synergistically enhance a more effective delivery of the active in the center.

One embodiment of the present invention is directed to confectionery products which are particularly adapted to deliver at least one active agent to infected and/or irritated throat tissues through the use of a core material surrounded by a hard outer shell. In this embodiment, the core contains at least one first active agent and is processed in a manner such that it contains substantially no gas (e.g. air.) If gas is present in the core, it can adversely affect the delivery of the active agent from the core region of the confectionery product, cause instability of one or more of the ingredients or actives and cause the incorrect amount of the active agent contained therein. Thus, in some embodiments of the present invention, the core, which is substantially void of gas, is capable of desirably delivering one or more active agents by delivering an accurate amount of active agent and minimizing or preventing possible degradation of the ingredients or active agents contained therein. The construction of the confectionery product enables more precise delivery of the active agents to a desired location, i.e. infected and/or irritated throat tissues.

The confectionery product also contains a hard outer shell which dissolves in the mouth and may optionally contain at least one active agent which may be the same or different than the active agent appearing in the core.

One embodiment of the present invention is generally directed to confectionery products which are capable of delivering at least one active agent to desired or targeted throat tissues which may be infected and/or irritated. Another embodiment of the present invention is directed to a lozenge comprising two active agents that can target certain tissues which may be infected and/or irritated.

The term “active agents” as used herein is intended to encompass agents other than food additives, which promote a structural and/or functional change in and/or on bodies to which they have been administered. These agents are not particularly limited, however, they should be physiologically acceptable and compatible with the lozenge. Useful active agents for the core and the outer shell include

(a) antimicrobial agents such as triclosan, cetyl pyridinium chloride, domiphen bromide, quaternary ammonium salts, zinc compounds, sanguinarine, fluorides, alexidine, octonidine, EDTA, and the like;

(b) non-steroidal anti-inflammatory drugs such as aspirin, acetaminophen, ibuprofen, ketoprofen, diflunisal, fenoprofen calcium, flurbiprofen sodium, naproxen, tolmetin sodium, indomethacin, celecoxib, rofecoxib, valdecoxib and the like;

(c) antitussives such as benzonatate, caramiphen edisylate, menthol, dextromethorphan hydrobromide, chlophedianol hydrochloride and the like;

(d) decongestants such as pseudoephedrine hydrochloride, phenylepherine, phenylpropanolamine, pseudoephedrine sulfate, ephedra and the like;

(e) antihistamines such as brompheniramine maleate, chlorpheniramine maleate, carbinoxamine maleate, clemastine fumarate, dexchlorpheniramine maleate, diphenylhydramine hydrochloride, azatadine maleate, diphenhydramine citrate, diphenylpyraline hydrochloride, doxylamine succinate, promethazine hydrochloride, pyrilamine maleate, tripelennamine citrate, triprolidine hydrochloride, acrivastine, brompheniramine, dexbropheniramine, fexofenadine, loratadine, desloratadine, fexofenadine, cetirizine, and the like;

(f) expectorants such as guaifenesin, ammonium chloride, ipecac, potassium iodide, terpin hydrate and the like;

(g) antidiarrheals such as loperamide and the like;

(h) histamine II receptor antagonists such as famotidine, ranitidine and the like;

(i) proton pump inhibitors such as omerprazole, lansoprazole and the like;

(j) general nonselective CNS depressants such as aliphatic alcohols, barbiturates and the like;

(k) general nonselective CNS stimulants such as caffeine, nicotine, strychnine, picrotoxin, pentylenetetrazol and the like;

(l) drugs that selectively modify CNS function such as phenyhydantoin, phenobarbital, primidone, carbamazepine, ethosuximide, methsuximide, phensuximide, trimethadione, diazepam, benzodiazepines, phenacemide, pheneturide, acetazolamide, sulthiame bromide, gabapentin, pregabalin, phenytoin and the like;

(m) antiparkinsonism drugs such as levodopa, amantadine and the like;

(n) narcotic-analgesics such as morphine, heroin, hydromorphone, metopon, oxymorphone, levorphanol, codeine, hydrocodone, xycodone, nalorphine, naloxone, naltrexone and the like;

(o) analgesic-antipyretics such salicylates, phenylbutazone, indomethacin, phenacetin and the like;

(p) psychopharmacological drugs such as chlorpromazine, methotrimeprazine, haloperidol, clozapine, reserpine, imipramine, tranylcypromine, phenelzine, lithium and the like;

(r) antifungals such as amphotericin and the like

(s) motion sickness treating agents such as hyoscine, prochloroperazine and the like

(t) local anesthetics such as benzocaine, lidocaine dyclonine, promoxine and the like

(u) antibiotics such as tyrothricin, amoxicillin, erthyromycin, cefalexin, azithromycin, ampicillin, tetracycline and the like

(v) nutraceuticals, vitamins, antiemetics such as ginger, ondansetron minerals, herbal products and the like

(w) antibacterial agents such as cetylpyridinium chloride, amylmetacreosol, thymol, benzalkonium chloride, chlorhexidine, hexylresorcinol and the like; and

(x) nicotine replacement agents for the treatment of addiction to smoking such as nicotine, cotinine and the like.

The pharmaceutically active agent is employed in an effective amount, which will vary depending, in part on the pharmaceutically active agent chosen. An “effective amount” is meant to be an amount of the pharmaceutically active agent that sufficient to at least reduce or relieve the condition, symptom or disease being treated, but low enough to avoid any adverse side effects. In addition to the particular active agent, the effective amount of the pharmaceutically active agent may vary with the type and/or severity of the disease, symptom or condition, the age and physical condition of the patient being treated, the duration of treatment, the nature of concurrent therapy, the specific form (i.e., salt) of the pharmaceutically active agent employed, and the particular carrier from which the pharmaceutically active agent is applied.

The amount of the pharmaceutically active agent in the formulation may be adjusted to deliver a predetermined dose of the pharmaceutically active agent over a predetermined period of time, which may typically vary from 1 to 24 hours.

Examples of doses for specific pharmaceutically active agents that can be delivered per one lozenge are reviewed in Table A.

TABLE A

| Pharmaceutically Active Agent | Dose | |
|---|---|---|
| Chlorpheniramine Maleate | 4 | mg |
| Brompheniramine Maleate | 4 | mg |
| Dexchlorpheniramine | 2 | mg |
| Dexbropheniramine | 2 | mg |
| Triprolidine Hydrochloride | 2.5 | mg |
| Acrivastine | 8 | mg |
| Azatadine Maleate | 1 | mg |
| Loratadine | 5-10 | mg |
| Phenylephrine Hydrochloride | 5-10 | mg |
| Dextromethorphan Hydrobromide | 10-30 | mg |
| Ketoprofen | 12.5-25 | mg |
| Sumatriptan Succinate | 35-70 | mg |
| Zolmitriptan | 2.5 | mg |
| Loperamide | 2 | mg |
| Famotidine | 10 | mg |
| Nicotine | 0.5-5 | mg |
| Diphenhydramine Hydrochloride | 12.5-25 | mg |
| Pseudoephedrine Hydrochloride | 15-30 | mg |
| cetyl pyridinium chloride | 0.5-10 | mg |
| Guaifenesin | 200-600 | mg |
| Cetirizine | 5-10 | mg |
| Nitroglycerine | 0.3-0.6 | mg |

Except as otherwise noted, the amount of active is designated as % by weight.

The lozenges may also be used to supply nutritionally acceptable components such as vitamins, minerals, trace elements, and fibers (preferably soluble fibers).

Examples of vitamins suitable for the incorporation in the composition of the invention include Vitamin A, Vitamin D, Vitamin E, Vitamin K, Vitamin C, folic acid, thiamin, riboflavin, Vitamin B (6), Vitamin B (12), niacin, biotin and panthotenic acid in pharmaceutical or nutritionally acceptable form. Examples of mineral elements and trace elements suitable for the incorporation in the composition of the invention include calcium, sodium, potassium, phosphorous, magnesium, manganese, copper, zinc, iron, selenium, chromium and molybdenum in pharmaceutical or nutritionally acceptable form. Useful amounts of a vitamin include from about 0.05 mg to about 3000 mg depending on the vitamin.

The term soluble fiber as used herein refers to fibers which are able to substantially undergo fermentation in the colon to produce short chain fatty acids. Examples of suitable soluble fibers include, carubin, pectin, tragacanth, cereal beta-glucan and the like. They may be hydrolysed or not.

In another embodiment of the present invention, the lozenge may further include one or more antimicrobial agents including, but not limited to, essential oils. Essential oils are volatile aromatic oils which may be synthetic or may be derived from plants by distillation, expression or extraction, and which usually carry the odor or flavor of the plant from which they are obtained. Useful essential oils may provide antiseptic activity. Some of these essential oils also act as flavoring agents. Useful essential oils include but are not limited to thymol, menthol, methyl salicylate (wintergreen oil), eucalyptol, carvacrol, camphor, anethole, carvone, eugenol, isoeugenol, limonene, osimen, n-decyl alcohol, citronel, a-salpineol, methyl acetate, citronellyl acetate, methyl eugenol, cineol, linalool, ethyl linalaol, safrola vanillin, spearmint oil, peppermint oil, lemon oil, orange oil, sage oil, rosemary oil, cinnamon oil, pimento oil, laurel oil, cedar leaf oil, gerianol, verbenone, anise oil, bay oil, benzaldehyde, bergamot oil, bitter almond, chlorothymol, cinnamic aldehyde, citronella oil, clove oil, coal tar, eucalyptus oil, guaiacol, lavender oil, mustard oil, phenol, phenyl salicylate, pine oil, pine needle oil, sassafras oil, spike lavender oil, storax, thyme oil, tolu balsam, terpentine oil, clove oil, and combinations thereof. In one embodiment the essential oils are selected from thymol, methyl salicylate, eucalyptol, menthol and combinations thereof.

One embodiment of the present invention includes the combinations of essential oils in LISTERINE® brand mouthwash and oral care strips as described in copending application Ser. No. 09/395,104, which is incorporated herein in its entirety. LISTERINE® brand mouthwash and oral care strips achieve their antimicrobial effect through such combination of essential oils that penetrate and kill the microorganisms. These essential oils include precisely balanced amounts of thymol, methyl salicylate, menthol and eucalyptol effective in killing the undesirable microorganisms.

Thymol ((CH₃)₂CHC₆H₃(CH₃)OH; isopropyl-m-cresol), also known by the chemical formula 5-methyl 2-(1-methylethyl) phenol, is an effective antimicrobial agent, and is typically obtained from the essential oil of Thymus vulgaris Labiatae and Monarda punctata Labiatae. Thymol is a white crystalline powder with an aromatic odor and taste and is soluble in organic solvents but only slightly soluble in deionized water.

Menthol (CH₃C₆H₉(C₃H₇)OH; hexylhydroxythymol) also possesses antiseptic properties and provides a cooling, tingling sensation. Menthol is isolated principally from the oil of Mentha arvensis. In its commercial form, menthol is available as L-menthol crystals obtained from a process involving cooling of the oil. Fractional distillation of peppermint oil, which usually contains from about 40% to about 65% menthol represents another important source of menthol. Synthetic sources of L-menthol are also available.

Eucalyptol (C₁₀H₁₈O; cineol), another essential oil with antiseptic properties, is derived from the eucalyptus tree. Eucalyptol is a terpene ether that provides a cooling, spicy taste and antiseptic activity. Having a camphoraceous odor and cooling taste, this essential oil is often combined with other essential oils such as menthol in confection formulations to impart medicinal effect.

Methyl salicylate (C₆H₄OHCOOCH₃), also known as wintergreen oil, is the main ingredient in many essential oils, constituting about 99% of oil of wintergreen (Gaultheria procumbens) and sweet birch (Betula lenta). Methyl salicylate is capable of providing flavoring and organoleptic flavor tones.

The essential oils may be used in amounts effective to provide biologic or therapeutic activity in the oral cavity. Generally, the total amount of essential oils present in the capsules or microcapsules can be from about 0.1% to about 50% w/w, optionally from about 0.5% to about 45%, or, optionally, from about 0.5% to about 10%.

Thymol is preferably employed in amounts of from about 0.001% to about 15% w/w, and optionally from about 0.01% to about 5% w/w. Eucalyptol is employed in amounts of from about 0.001% to about 15% w/w, and optionally from about 0.01% to about 5% w/w. Menthol is employed in amounts from about 0.1% to about 25% w/w, most preferably from about 0.1% to about 15% w/w. Methyl salicylate is employed in amounts of from about 0.001% to about 15% w/w, and optionally from about 0.01% to about 10% W/W.

The amounts added can be readily determined to those skilled in the art and can exceed these amounts as long as the total oil content does not create processing problems. In certain embodiments, the essential oils are combined in amounts synergistically effective to kill the plaque-producing germs that cause dental plaque, gingivitis and bad breath.

As used herein, the term “core material” includes the combination of a carrier material and an active agent. The term “carrier material” are those ingredients which when combined with the active agent form the core material. The term “target tissues” means tissues of the oral cavity and especially tissues of the lower throat which desirably benefit from the delivery of the active agents by the confectionery products of the present invention.

The core is made of a carrier material which is in the form of a liquid-like to a gel-like state when exposed to the oral cavity and is capable of traveling with saliva produced in the oral cavity, at least in part as the result of the presence of the confectionery product, to the targeted areas of the throat. Prior to delivery of the lozenge to the oral cavity, the core may be in a liquid-like, gel-like to a solid state that may have a low viscosity. The core may have a viscosity from about 1 to about 100,000 cps. Once administered to the buccal cavity, the heat of the oral cavity warms the core so that it is in a more viscous liquid like to gel-like state thereby enabling delivery of the active agents to the targeted area. In one embodiment, the core material forms a liquid when exposed to the oral cavity. The core material is typically present in an amount of from 20 mg to 3000 mg, preferably 250 mg to 2000 mg.

The core and the shell may include any material, which is compatible with the formation of a carrier material suitable for delivery of the active agent to the targeted tissues of the throat. In particular, useful materials include sweeteners including sugars such as sucrose, corn syrup and the like and sugarless sweeteners such as polyols e.g. isomalt, maltitol, sorbitol and the like; emulsifiers such as lecithin and the like taste masking agenst, such as aluminum magnesium silicate and the like; and fats and oils such as medium chain triglycerides, such as Neobee 1053 as sold by Stepan may be used in the core and/or the shell. Flavorings such as menthol, eucalyptol, strawberry flavorings such as those sold by Firmenich and the like. Additional details and examples of these materials are described in copending application Ser. No. 09/395,104, which is incorporated herein in its entirety.

The amount of the first active agent contained within the core material is in an amount sufficient to provide the desired effect when released from the core upon exposure to the temperature of the oral cavity. A typical amount of the active agent will be in the range of up to about 1000 mg, preferably from about 0.1 mg to 750 mg based on the total weight of the core material. The amount of the first active agent will depend in part on the active agent selected, the size of the core and the composition of the core material.

In accordance with the present invention, the core is surrounded by the shell within the confectionery product in a manner such that there is little if any leaking of the core material into the shell and outside of the confectionery product prior to dissolution. It is desired that the core be provided in a definable shape and is visible through the shell. By being visible through the shell of the confectionery product, the resulting product has a visible display of the core containing an active agent.

The core may be provided in any shape. Such shapes include conventional shapes such as spheres, cubes, stripes, and the like, including a plurality of the same as well as less conventional or eccentric shapes, such as cartoon characters, polygons, symbols (e.g. a letter of the alphabet) and the like. The ability to fashion the core in a variety of shapes can facilitate use of a lozenge to administer active agents to children (e.g. when the core is in the shape of a cartoon character). Furthermore, the shape of the core may be identified with a particular active agent e.g. a square shape may indicate the presence of a particular antibiotic).

The amount of the core material relative to the confectionery product will typically be in the range of up to 75% by weight, more typically from about 10 to about 25% by weight based on the total weight of the confectionery product.

The shell material is a solid material, typically a hard candy comprised of conventional shell forming materials including sugars ( e.g. sugar, corn syrup and the like), non-sugar sweeteners such as sugar alcohols (e.g. isomalt alone or in combination with high intensity sweeteners such as aspartame, neotame, sucralose, saccharin, acesulfame potassium salt and the like), flavorants, acidulants, cooling compounds, coloring agents and the like as described in copending application Ser. No. 09/395,104, which is incorporated herein in its entirety. The shell when placed in the oral cavity should trigger salivation and be dissolved within a typical amount of time for the dissolution of confections within the oral cavity.

In one embodiment the shell material includes at least one second active agent which may be the same or different than the first active agent contained within the core and generally may include any of the active agents previously described. In some cases, agents included within the core and the shell, respectively might be incompatible such as a local anesthetic (e.g. amethocaine) and an antibacterial agent (e.g. chlorlexidine), an expectorant (e.g. ammonium chloride) and an antitussive (e.g. codeine), a local anaesthetic (e.g. lidocaine) and an antifungal (e.g. amperotericin) and the like. In some cases, certain excipients may be incompatible with other excipients or actives, such as some honey-lemon flavoring ingredients interact with some active agents, such as benzocaine. In particular, aldehyde sugars can cause hydrolysis of ester compounds, such as benzocaine. Accordingly, an embodiment of the present invention provides for a lozenge that is capable of containing incompatible excipients or active agents. In particular, one embodiment prevents interaction of incompatible components in the lozenge by placing incompatible ingredients separate from each other in the shell or the core.

In another embodiment, the core and shell may contain at least one vaporizable active agent such as, for example, essential oils (e.g. thymol, menthol, and the like). The vaporizable agent which is an optional feature of the present invention can be used to provide relief from congested and/or infected nasal passages in addition to delivering an active agent.

The amount of the active agent contained within the shell will typically be up to 30% by weight, more typically from about 1 to 25% by weight based on the total weight of the shell material.

Other materials that may be added to the core material and/or the shell material include viscosity modifying agents, taste masking agents, demulcents (i.e. throat coating agents) and the like.

Suitable examples of carriers include medium chain triglycerides, glycerin, emulsifiers (e.g. lecithin, polysorbate 80, mono and diglycerides and the like), propylene glycol, benzyl alcohol, triacetin, and combinations thereof as well as other carrier agents described in copending application Ser. No. 09/395,104, which is incorporated herein in its entirety.

Suitable examples of viscosity modifying agents include medium chain triglycerides, glycerin, emulsifiers (e.g. lecithin, polysorbate 80, mono and diglycerides and the like), propylene glycol, benzyl alcohol, triacetin, carboximides, and combinations thereof as well as other viscosity modifying agents described in copending application Ser. No. 09/395,104, which is incorporated herein in its entirety.

Useful taste masking agents include fats and oils (e.g. partially hydrogenated cottonseed oil, hydrogenated coconut oils), essential oils (e.g. menthol, eucalyptus oil and the like) and cooling agents such as WS-3 sold by Wilkinson Sword.

Useful demulcents include pectin, glycerin, gelatin and gums such as carrageenan, guar and gellan and the like.

As previously indicated, the confectionery products are constructed in a manner in which the core is surrounded by the shell material. In an embodiment of the invention, the shell material is either transparent or translucent allowing the core to be visible therethrough which provides the added feature “showing” the user the presence of the core material containing an active agent and can therefore provide a designated shape matched to a particular active agent as previously described. In another embodiment of the invention, the shell material is essentially clear (i.e. transparent or translucent and colorless) to provide the greatest contrast between the shell material and a colored core material. The core material may be any color depending on the selection of a food grade suitable coloring agent. Examples of typical coloring agents include FD&C Red 40, FD&C Blue 2, Carmine, FD&C yellow 5, beta carotene and the like.

A shell which enables the core to be seen therethrough can be prepared, for example, in the following manner. An isomalt slurry is quickly heated such as by microfilm cooking techniques. Quick cooking (e.g. for about 5 minutes) minimizes browning of the Isomalt thus creating a very clear shell. A small of amount of coloring agent or citrus acid may be added if desired.

The confectionery products of the present invention will most typically be in the form of a lozenge or a hard sucking candy but may include lollypops, and any other shaped or formed product which can be formed from a core material and a shell material in accordance with the present invention.

The confectionery products of the present invention can be prepared in a variety of processing technologies including double depositing, hand-pressing, rotary forming and extrusion. Such techniques are well known in the art such as disclosed in Sugar Confectionery Manufacture, 2^{nd} Edition, Edited by E. B. Jackson (1995). In an embodiment of the invention, the confectionery product is made by separately combining the ingredients of the shell material and core material in a vessel and then delivering a stream of the respective materials to a manifold which provides for the interruptible flow of the core material and a continuous flow of the shell material surrounding the core material. The resulting product is ejected in discrete units corresponding to the desired weight and size of the confectionery product and placed in trays with individual compartments for storing the confectionery products until they cool to ambient temperature.

In one embodiment of the present invention, the core material is degassed. Degassing techniques remove air from the core material thus at least minimizing chemical reactions therein. The core material can be prepared in an enclosed mixing vessel and processed under vacuum. Alternatively, the core materials are combined and mixed together and then a vacuum is applied to the mixture to remove any gases contained therein.

A process for forming the confectionery product will ensure that the core material is directly injected within the shell material. One such valve system is a manifold system, which may employ a ball/stall or ball/spring valve assembly. This ensures that the core material is completely surrounded by the shell material and allows the core to be deposited within the final product (e.g. lozenge).

The process is typically temperature controlled with a series of heaters/coolers shown sufficient to maintain the shell material at a temperature of from about 1° C. to about 200° C. and the core material from about 1° C. to about 200° C. which is a temperature sufficient to maintain the core material centrally positioned within the shell material and to enable the same to be ejected as discrete units of the confectionery product.

### EXAMPLES

### Method of Preparation

### Shell Preparation

The preparation of the shell material for forming the confectionery product by mixing hydrogenated isomalt (Isomalt from Palatinit of America) and water in a suitable vessel under heating to about 165° C. to form a candy base. A small amount of citric acid was added to the vessel. The candy base is then cooled to about 145° C. enable the addition of a suitable sweetener (e.g. a high intensity sweetener such as aspartame, neotame and the like), an optional active agent and flavors and any other suitable ingredients.

### Center Preparation

The core material may be prepared by mixing maltitol syrup (Lycasin 80/55 from Roquette America) and a colorant, if desired, in a suitable vessel under heating to form a candy base. The candy base is then cooled to about 70° C. or lower to enable the addition of a suitable viscosity modifying agent, such as glycerin, sweetener (e.g. high intensity sweetener) the active agent, a flavorant and any other suitable ingredients.

The respective shell and core materials are then added to separate hoppers which materials are then combined as previously described.

### Example 1

### Centerfilled Lozenge with 10-mg Benzocaine and 1.4-mg CPC in Center

A center-filled lozenge having a core containing 10-mg benzocaine and 1.4-mg cetyl pyridinium chloride (CPC) was prepared according to the above Method of Preparation and had a formulation as specified in Table 1. The total weight of the lozenge was about 4.5 grams.

TABLE 1

| Ingredients | % by weight |
|---|---|
| Shell | |
| Isomalt | 82.67 |
| Citric Acid | 0.05 |
| Center | |
| Lycasin | 11 |
| Glycerin | 3 |
| Benzocaine | 0.22 |
| Cetyl pyridinium Chloride | 0.03 |
| Lecithin | 0.002 |
| Center and Shell | |
| Residual Moisture* | 2.5 |
| Strawberry Flavor | 0.2 |
| Menthol | 0.2 |
| Eucalyptol | 0.1 |
| Aspartame | 0.05 |
| Acesulfame Potassium Salt | 0.03 |

*Residual moisture refers to the amount of moisture estimated to remain after cooking the Isomalt and water mixture in the Shell Preparation and the Lycasin in the Center Preparation.

### Example 2

### Center-filled Lozenge with 200-mg Guaifenesin in Shell, 20-mg Dextromethorphan in Center

A center-filled lozenge containing 200-mg guaifenesin in Shell, 20-mg dextromethorphan in center composition was prepared according to the above Method of Preparation and had a formulation as specified in Table 2. The total weight of the lozenge was about 4.5 grams.

TABLE 2

| Ingredients | % by Weight |
|---|---|
| Shell | |
| Isomalt | 79.015 |
| Citric Acid | 0.05 |
| Center | |
| Lycasin | 10 |
| Guaifenesin | 4.4 |
| Glycerin | 3 |
| Dextromethorphan | 0.5 |
| Lecithin | 0.002 |
| Center and Shell | |
| Residual Moisture* | 2.5 |
| Strawberry Flavor | 0.2 |
| Menthol | 0.2 |
| Eucalyptol | 0.1 |
| Aspartame | 0.05 |
| Acesulfame Potassium Salt | 0.03 |

*Residual moisture refers to the amount of moisture estimated to remain after cooking the Isomalt and water mixture in the Shell Preparation and the Lycasin in the Center Preparation.

### Example 3

### Center-filled Lozenge Containing 5 mg of Nicotine in Shell and Essential Oils in Center.

A center-filled lozenge containing a shell having 5 mg of nicotine and a center containing Listerine essential oils was prepared according to the above Method of Preparation and had a formulation as specified in Table 3. The total weight of the lozenge was about 4.5 grams.

TABLE 3

| Ingredient | % by Weight |
|---|---|
| Shell | |
| Isomalt | 84.079 |
| Nicotine | 0.02 |
| Citric acid | 0.05 |
| Menthol (added as a flavoring) | 0.2 |
| Eucalyptol (added as a flavoring) | 0.1 |
| Center | |
| Lycasin | 9.00 |
| Glycerin | 3.00 |
| Thymol | 0.09 |
| Methyl Salicylate | 0.11 |
| Eucalyptol | 0.13 |
| Menthol | 0.19 |
| Lecithin | 0.02 |
| Center and Shell | |
| Residual Moisture* | 2.5 |
| Strawberry Flavor | 0.2 |
| Neobee 1053 (Medium Chain | 0.40 |
| Triglycerides) | |
| Aspartame | 0.05 |
| Acesulfame Potassium Salt | 0.03 |

*Residual moisture refers to the amount of moisture estimated to remain after cooking the Isomalt and water mixture in the Shell Preparation and the Lycasin in the Center Preparation.

While the invention has been explained by a detailed description of certain specific embodiments of it, it is to be understood that various modifications and/or substitutions may be made without departing from the spirit of the invention. Accordingly, the invention should not be deemed limited by the detailed description of the embodiments set out above, but only by the following claims appended hereto.

1. A confectionery product for the delivery of at least one pharmaceutically active agent to a targeted tissue of the throat comprising:
a) a core comprising a carrier material; said carrier material when exposed to the oral cavity being in liquid form suitable for contacting the targeted tissues of the throat, and at least one first pharmaceutically active agent suitable for treating said targeted tissues;
b) a shell comprising a solid material suitable for dissolving in the oral cavity
c) said core positioned within the shell; and
d) said core being substantially void of gas.

2. The confectionery product of claim 1 wherein the core is visible through the shell.

3. The confectionery product of claim 1 wherein the core minimizes degradation of the active agents and delivers an accurate amount of active agent to a consumer.

4. The confectionery product of claim 1 wherein the shell contains at least one pharmaceutically active agent.

5. The confectionery product of claim 4 wherein the first and second active agents are independently selected from the group consisting of antitussives, local anesthetics, nutraceuticals, vitamins, antiemetics, antihistamines, cold treating agents, motion sickness treating agents, anti-fungals, antibiotics, antibacterial agents, expectorants, constipation treating agents, decongestants, essential oils, herbal products, nicotine replacement agents and combinations thereof.

6. The confectionery product of claim 5 wherein said pharmaceutically active agent is selected from the group consisting of benzocaine, hexylresorcinol, benzalkonium chloride, dextomethorphan, guaifenesin, cetyl pyridinium chloride and combinations thereof.

7. The confectionery product of claim 5 wherein said active agents are in amounts from about 1 to about 500 mg.

8. The confectionery product of claim 1 wherein the core material is present in an amount of from about 250 to 900 mg.

9. The confectionery product of claim 1 where the shell comprises a vaporizable active agent.

10. The confectionery product of claim 9 wherein said vaporizable agent is selected from the group consisting of menthol, eucalyptol and combinations thereof.

11. The confectionery product of claim 1 wherein the color of the core is different than the color of the shell.

12. The confectionery product of claim 1 in a form selected from the group consisting of lozenges, lollipops and hard candies.

13. A method of producing a confectionery product for delivering at least one pharmaceutically active agent to targeted tissues of the throat, said confectionery product comprising a core and a shell, said core containing a carrier material having a physical form ranging from a liquid to a solid, said carrier when exposed to the oral cavity being in a liquid form for contacting the targeted tissues of the throat, and at least one first pharmaceutically active agent suitable for treating said targeted tissues, said shell comprising a solid material suitable for dissolving in the oral cavity and optionally comprising at least one second active agent, said method comprising:
a) combining the carrier material with said first active agent to form a core material in a first vessel;
b) removing any gas contained within the core material;
c) forming a shell material containing the optional second active agent in a second vessel;
d) injecting an interruptible stream of the core material into a conduit while simultaneously injecting a continuous stream of the shell material external of the core material; and
e) intermittently ejecting the combined stream in the form of said confectionery product.

14. The method of claim 13 comprising ejecting the combined streams in the form of said confectionery product into a tray containing a plurality of individual confection receiving compartments and allowing the confectionery product to cool therein to ambient temperatures.

15. A confectionery product for the delivery of at least two pharmaceutically active agents to a targeted tissue of the throat comprising:
a) a core comprising a carrier material; said carrier material when exposed to the oral cavity being in liquid form suitable for contacting the targeted tissues of the throat, and at least one first pharmaceutically active agent suitable for treating said targeted tissues;
b) a shell comprising a solid material suitable for dissolving in the oral cavity; said shell comprising at least one second pharmaceutically active agent;
c) said core positioned within the shell; and
d) said core being substantially void of gas.
